(19) United States
(12) Patent Application Publication
(10) Pub. No.: US 2014/0171912 A1
(43) Pub. Date: Jun. 19, 2014

(54) DRUG-ELUTING ANGIOPLASTY BALLOON SYSTEMS

(71) Applicant: CALIFORNIA INSTITUTE OF TECHNOLOGY, Pasadena, CA (US)

(72) Inventors: Morteza Gharib, Altadena, CA (US); Hesham Azizgolshani, Winnetka, CA (US)

(73) Assignee: CALIFORNIA INSTITUTE OF TECHNOLOGY, Pasadena, CA (US)

(21) Appl. No.: 14/109,662

(22) Filed: Dec. 17, 2013

Related U.S. Application Data
(60) Provisional application No. 61/902,954, filed on Nov. 12, 2013.
(60) Provisional application No. 61/859,103, filed on Jul. 26, 2013.
(60) Provisional application No. 61/832,622, filed on Jun. 7, 2013.
(60) Provisional application No. 61/830,813, filed on Jun. 4, 2013.
(60) Provisional application No. 61/738,892, filed on Dec. 18, 2012.

(51) Int. Cl. A61M 25/10

(52) U.S. Cl. CPC A61M 25/104
USPC 604/509; 604/103.02

(57) ABSTRACT

Systems, devices, and methods are provided for drug-eluting angioplasty balloons. An underlying balloon core member is protected by a core-screen from mechanical and flow shear forces during delivery. Inflation of the balloon opens the screen and pores in the screen permitting drug transfer and absorption. Upon deflation, the screen can be compressed and withdrawn with the balloon.

### RELATED APPLICATIONS

This application claims the benefit of and priority to U.S. Provisional Patent Application Ser. No. 61/738,892, filed Dec. 18, 2012, U.S. Provisional Patent Application Ser. No. 61/830,813, filed Jun. 4, 2013, U.S. Provisional Patent Application Ser. No. 61/832,622, filed Jun. 7, 2013, U.S. Provisional Patent Application Ser. No. 61/859,103, filed Jul. 26, 2013, and U.S. Provisional Patent Application Ser. No. 61/902,954, filed Nov. 12, 2013, every one of which is incorporated by reference herein in its entirety for all purposes.

### FIELD

The subject matter described herein relates to systems, devices, and methods for Drug-Eluting Balloon (DEB) or Drug-Coated Balloon (DCB) angioplasty.

### BACKGROUND

Two different classes of medical devices bear superficial similarity to the subject devices. The first class of devices are fiber-reinforced balloons (e.g., U.S. Pat. Nos. 4,637,396; 5,868,779; 6,746,425 and 7,252,650 and US Publication Nos. 2002/0161388; 2009/0038752; 2011/0172698 and 2012/0296363) adapted for intraluminal use, including Percutaneous Transluminal Coronary Angioplasty (PTCA). In numerous variations, braids, knits and other mesh are incorporated in the design in order to provide burst strength and/or limit balloon expansion. The second class of devices are stent-type implants (e.g., EP Patent 0 895 761 and U.S. Pat. Nos. 5,383,982 and 5,817,126) incorporating braids that are set over PTCA balloons for delivery and deployment upon balloon expansion.

DEB technology is garnering significant clinical and market interest in view of its potential advantages in terms of protecting against late lumen loss without the disadvantages associated with stent (particularly, drug-eluting stent) use. See, for example, Waksman R., et al., “Drug-Eluting Balloon: the Comeback Kid?” Circulation: Cardiovascular Interventions, 2009; 2: 352-358, Drug-Eluting Balloon. Still, of the several commercially available DEB systems employing a coating set upon a balloon, significant issues remain with inadvertent drug loss during delivery to the treatment site.

These existing systems can fail to adequately protect their drug coating from peeling and breaking free during advancement to the treatment site, and thus exposing the systemic circulation to potent drugs prematurely and potentially leading to embolism and other complications. As such, some of the freed drug is washed-out in the form of particulates during vascular travel. To compensate for the drug lost during deployment, an excess amount of the drug is generally applied on the balloon surface, but this can lead to exposure to unnecessary levels of the drug and can even pose risk of overdose. Accordingly, needs exist to address these issues in DEB and DCB systems.

### SUMMARY

Provided herein are a number of embodiments of systems, devices, and methods that variously meet the above-referenced needs via an approach that protects drugs from both washout and/or overdose. In certain embodiments, a Percutaneous Transluminal Angioplasty (PTA) device is formed by covering a drug-loaded angioplasty balloon with a fine mesh flexible sleeve. The so-called “mesh” or “screen” may include a braided sleeve made out of Nylon filaments or wires (as further shown and described) or out of bio-compatible polymer filaments. Alternative filament materials such as NiTi, CoCr, or Stainless Steel may be employed in constructing the sleeve. Likewise, biodegradable materials can be used in manufacturing the screen, preventing any complications that may arise if the screen itself gets damaged during drug delivery process. Moreover, though reference is made to PTA-type devices, it is to be understood that the present embodiments may be used in other situations and/or contexts. Likewise, alternative methods of treatment are possible as well, including, for example, treatment in the urinary tract or for colon cancer.

Structurally, the sleeve overlays or jackets and thereby screens-off a DEB core member. This “core-screen” is minimally porous when non-expanded (or in its contracted state) and highly porous when fully expanded. The core-screen can, in many embodiments, be used with a drug that has a tendency to break or peel off from the balloon in the form of particulates, such as flakes. The core-screen protects the drug (e.g., Paclitaxel) on the surface of the deflated balloon from breaking off due to mechanical frictional forces arising from, for example, the vessel wall and introducer (guide catheter) wall (in the case of contact) and fluid shear forces (in the case of blood flow). In a deflated state or condition, the drug-coated balloon surface is covered by the screen, thereby minimizing (vessel and/or delivery or guide catheter) wall contact and thus the loss of drug during balloon travel or tracking towards the target location.

In certain embodiments, the core-screen is optionally tensioned or “pulled” into a minimum diameter configuration for device tracking. In other embodiments, there is no mechanism for tensioning and the mesh may be regarded as (relatively-speaking) “loose” in its minimal diameter configuration.

The embodiments described herein effectively prevent drug peel off during vascular travel that could enter the blood stream as undesirable floats. In many embodiments, the screen, in the contracted state, protects the balloon and substantially prevents (e.g., reduces to a clinically insignificant level) the passage or transfer of the drug therethrough (as the spaces of the screen are too small). Upon inflation of the balloon at the target location, the expanded balloon will contact the mesh surface and force it to expand into contact with a region of the luminal wall. In the expanded state, the spaces of the screen are enlarged enough to allow efficient transfer of the drug to the arterial plaque (or other tissues) by forced mechanical contact and/or diffusion transfer.

For example, each space of the screen, when in the contracted state, can be smaller than the typical or average particulate size of the drug when broken, peeled, or freed from the balloon surface. When in the at least partially expanded state, each space of the screen can be larger than the typical or average particulate size of the drug when broken, peeled, or freed from the balloon surface. The screen can be adapted such that the point (of radial expansion of the screen) where the spaces in the screen become large enough to permit drug transfer occurs at or before the point where the screen makes contact with the surrounding vasculature, or the point where the balloon (or other expandable structure with drug delivery capability) is fully inflated or otherwise inflated to the appropriate radial dimension for drug delivery.

In many example embodiments, a braided sleeve (e.g., constructed from filaments, wires, ribbons, or threads, etc.) is provided that is unsecured to the balloon and/or underlying drug coating, and the individual filaments that are interlaced, interwoven or intertwined within the mesh can move freely upon expansion. In some examples, the braided sleeve is heatset so that the mesh it defines is stable and its filaments (largely) pivot when moving relative to one another. In other examples, the filaments can both pivot and translate relative to one another when moved (at least in part) by expansion of the balloon. In some embodiments, if only a portion of the surface area of the balloon is coated with the drug, such as for more targeted delivery, the sleeve can be configured to cover only that corresponding portion of the balloon surface area, with a manner of attachment that maintains the coupling of the screen to the balloon.

In some examples, the core-screen is a braided construction. Such a construction advantageously transitions between a reduced (or minimum width, or contracted) configuration and an enlarged (or full width, or expanded) configuration without need for significant flex or elongation of the filaments defining the structure. Other constructions may be employed in instances where substantially elastic filaments are employed to allow for elastic expansion (as opposed to a configuration change as with the use of inelastic braid). One example that may be employed for the core-screen is a plurality of “knits” including a series of interlocking loops. Another example is a mesh comprising a braid that interlaces or knots inelastic filaments (lengthwise) over and under circular, elastic bands (circumferential).

However configured, the core-screen material does not impose mechanical breaking of the coated drug during vascular travel or expansion processes. Moreover, the present embodiments allow the screen openings to be maximized in size upon inflation of the balloon.

Overall, an attractive feature of the present embodiments is an independence from the coated drug or coating technique used. Therefore, the core-screen approach can be adopted for different drug coatings.

Various architectures for achieving the aforementioned results are described. In connection with such description, other systems, devices, methods, features and advantages of the subject matter hereof will be or will become apparent to one with skill in the art. It is intended that all such additional systems, devices, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIGS. 1A and 1B are side and end view schematics, respectively, of an example embodiment of a DEB with a core-screen in a deflated state at a treatment site.

FIGS. 2A and 2B are side and end view schematics, respectively, of an example embodiment of a DEB and core-screen in an inflated state at the treatment site.

FIGS. 3A and 3B are detailed photographs of an example embodiment of the core-screen over a balloon in deflated and inflated states.

FIGS. 4A-4C are photographs of example embodiments of currently-available DEBs that may be incorporated in embodiments hereof.

FIG. 5 illustrates the component parts for an example embodiment of a core-screen DEB.

FIGS. 6A and 6B illustrate the delivery and deployed states of an example embodiment of a core-screen DEB incorporating a spring as an extensible element.

FIGS. 7A and 7B illustrate the delivery and deployed states of another example embodiment of a core-screen DEB incorporating a compression spring.

FIGS. 8A and 8B illustrate the delivery and deployed states of another example embodiment of a core-screen DEB including fixed sleeve ends.

FIGS. 9A and 9B illustrate the delivery and deployed states of another example embodiment of a core-screen DEB including a remote sleeve-retraction setup.

### DETAILED DESCRIPTION OF EMBODIMENTS

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

This disclosure describes various embodiments that can be employed to protect the drug on the surface of drug eluting balloons or other expandable drug eluting medical devices during deployment and retraction. These embodiments correspond to keeping a sleeve-type protective mesh with minimal open area in place over a drug coated balloon surface during deployment as the catheter balloon is tracked to the location of interest by those skilled in the art, as well as embodiments that restore the balloon and the protective mesh screen after deployment to a collapsed state during the retraction process. As the balloon or expandable drug eluting medical device is inflated or expanded, the open area of the protective mesh is increased and the drug becomes accessible to be delivered to the site of interest. The mesh described here can be made in a variety of forms and materials including but not limited to plastics, metals, or their combinations and manufactured through a variety of manufacturing methods known to those knowledgeable in the art including, but not limited to, braiding and extrusion based methods.

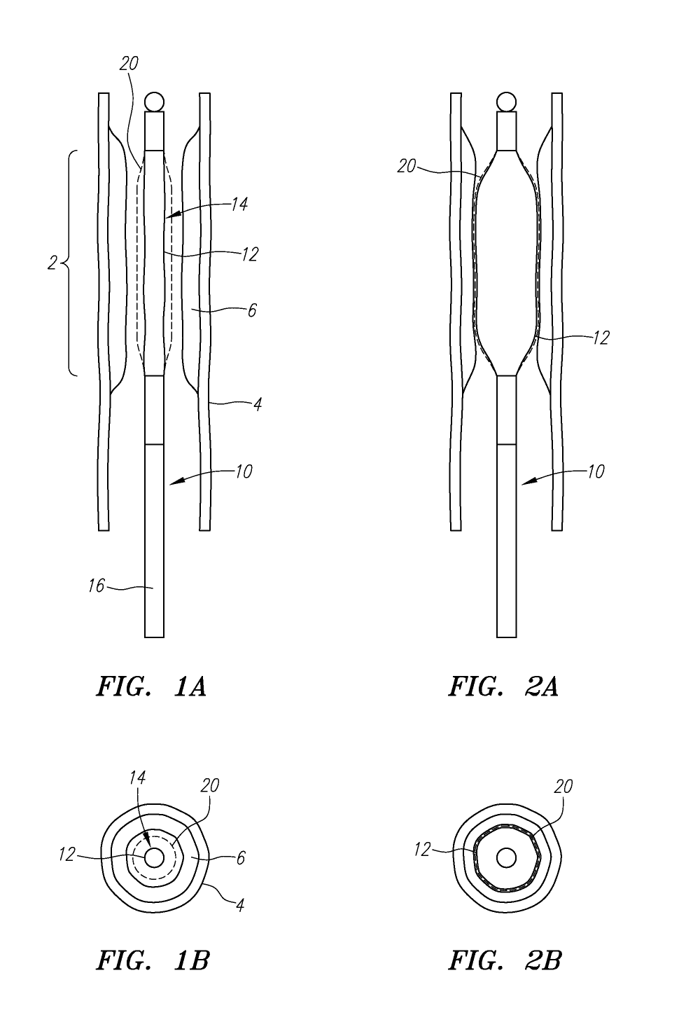

Turning now to FIGS. 1A-2B, the subject system or device 10 is shown at a target location or site 2. The exemplary target location may be in a coronary or a peripheral artery that includes a vessel lumen defined by a wall 4 and a lesion depicted by a layer of plaque 6.

In FIGS. 1A and 1B, device 10 includes a balloon 12 (e.g., the DEB) and a screen 20. Balloon 12 has a drug coating or layer 14 adhered or affixed located thereon (e.g., by dip, spray or otherwise). Device 10 is covered by screen 20 in the form of a sleeve, which is shown in a minimal diameter or delivery configuration. Device 10 can be advanced through proximal vasculature over a guidewire and/or through an introducer or guide by a physician pushing a shaft section 16 (such features/action not shown). The shaft and any associated parts may be configured in a so-called “Rapid Exchange” configuration if desired.

As shown in FIGS. 2A and 2B, balloon 12 is inflated (e.g., by a manual endoflator, not shown) so that the screen contacts the plaque. It may, in part, compress the plaque whereas the balloon is intended to primarily serve that purpose.

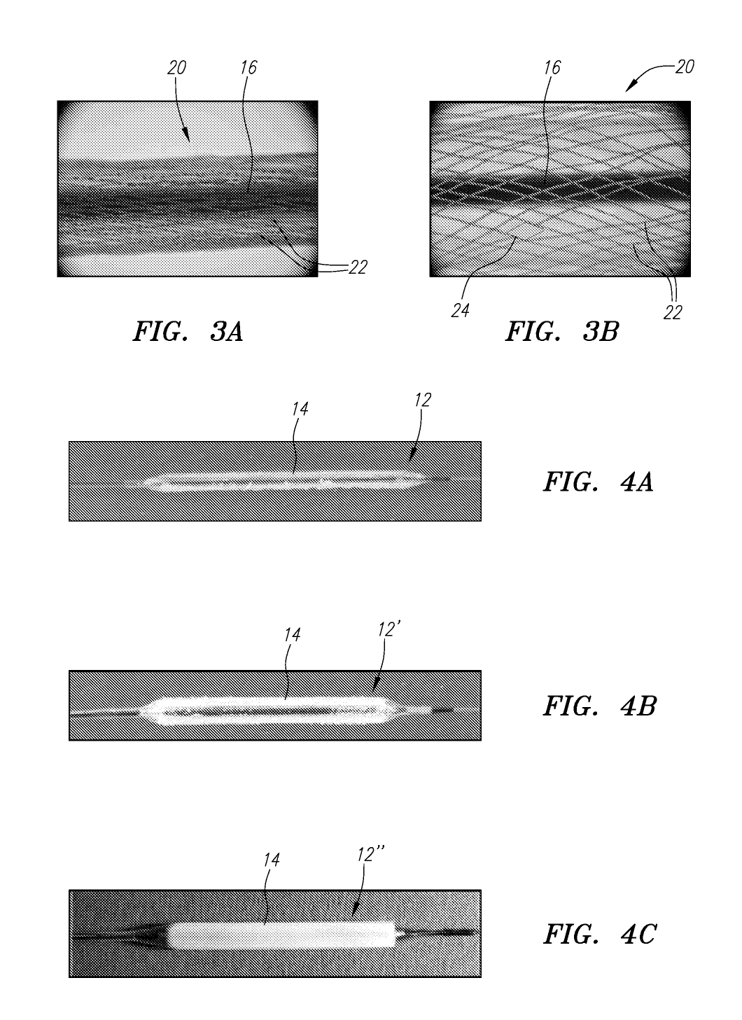

Regardless, the balloon pushes the screen radially outward causing it to expand as shown in FIGS. 3A and 3B. Here, screen 20 includes a braided Nylon sleeve in which its constituent fibers 22 are about 75 μm in size and braided in a two-over-two interlace pattern. Other braid patterns (e.g., one-over-one), materials (e.g., stainless steel, nitinol, etc.), various filament “end” counts in the braid (e.g., from about 48 to about 144 or more), and filament diameters (e.g., from about 25 to about 100 μm size) may be employed. As for the filament size, such a range offers the filaments adequate strength to braid and coverage, without being so large as to space the underlying balloon too far from the vessel wall, plaque, or other target site into which drug is to be delivered.

In FIG. 3A, core-screen 20 is shown at its minimum diameter with the braid from which it is constructed in a “jam” condition with minimal to zero pore size. In FIG. 3B, screen 20 is expanded over an inflated balloon 12 (where the underlying balloon is transparent, but shaft 16 is visible). As such, spaces, gaps, interstices or pores 24 (of uniform or non-uniform configuration) develop through which drug transfer from the balloon surface to the target site may occur essentially unimpeded. Stated differently, spaces 24 provide ports or pathways for drug delivery.

According to one method for enhanced drug delivery, the balloon is at least partially deflated with the drug delivery is intended to occur. Due to elastic (or superelastic) properties of core-screen fibers 22 and/or their configuration, screen 20 may stay in contact with the lesion. When balloon 12 is at least partially contracted, a chamber is then defined between the expanded screen 20 and the balloon wall. This chamber is more closed toward each end in the case of a braid-based construction of screen 20. Drug is able to circulate within this chamber and be evenly distributed and absorbed by tissues at the lesion.

In any case, the selected drug for the DEB may be paclitaxel together with any desired carrier or matrix (e.g., a hydrophilic iopromide-derived carrier) and any coating or loading approach may be employed. Moreover, balloon 12 may be constructed similarly or identically to any of a variety of commercially-available DEB designs such as represented in FIGS. 4A-4C. FIG. 4A depicts an IN.PACT AMPHIRION balloon 12 from Invatec, FIG. 4B depicts a SEQUENT balloon 12′ from BBraun, and FIG. 4C depicts an ELUTAX balloon 12″ from Aachen Resonance, and each have various applications of drug 14.

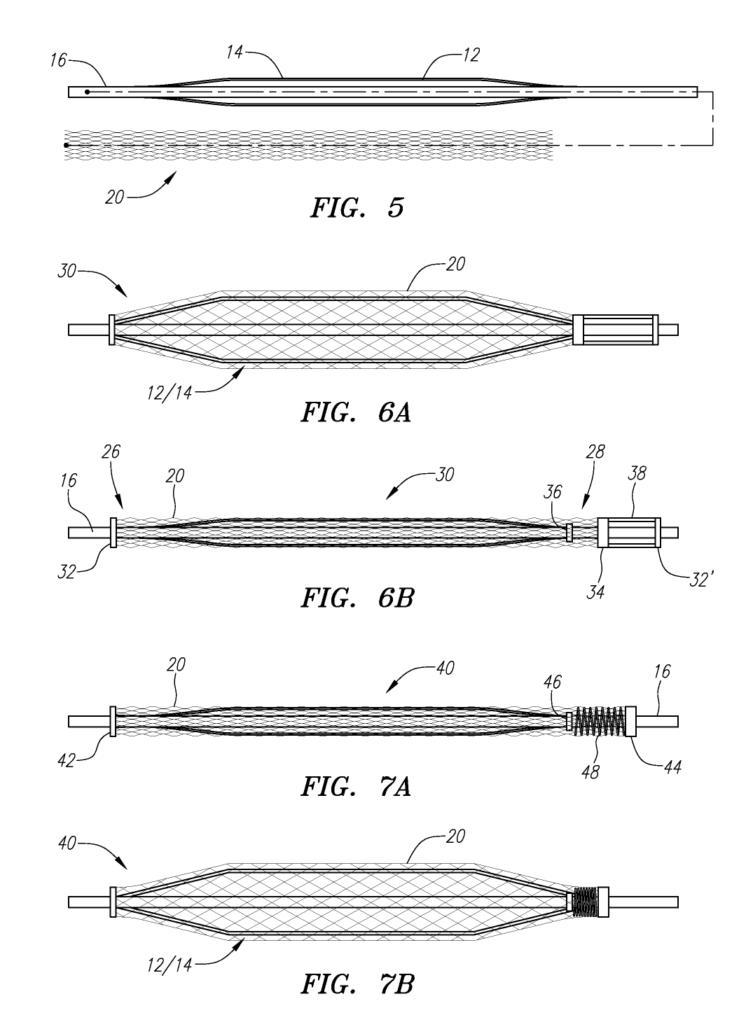

FIG. 5 is an assembly view illustrating the integration of such an exemplary balloon 12 and screen 20 to operate as device 10. Notably, in many of the embodiments herein, balloon 12 requires no reinforcement for use. In other words, screen 20 need not or may not increase the burst pressure or limit the distention of the balloon assembly. Rather, balloon 12 can optionally be of the “non-compliant” (or inelastic, or substantially inelastic) variety (i.e., as constructed of blown PET as typical to many PTCA balloons). Also, various features and methods can be employed to keep screen 20 from releasing from the balloon surface, either by sliding distally or proximally or by spacing apart radially from the balloon surface.

FIGS. 6A and 6B depict an example embodiment of device 30, in contracted and expanded states, respectively. Here, a fixed ring or band element 32 holds a first end 26 of mesh screen 20 fixed with respect to catheter shaft 16. This fixed ring element 32 may be one or a combination of crimped metal rings, heat shrink plastic, or adhesives applied in a circumferential manner that achieves the desired function of affixing one end of screen 20 to catheter shaft 16. A second end 28 of screen 20 is then advantageously attached to a mobile ring 34 that allows shortening or lengthening of screen 20 upon inflation and/or deflation of balloon 12. Mobile ring 34 can be of different or similar materials as screen 20 or, in one particular embodiment, can be an extension of the mesh screen braided differently at the end. The fixed ring 32 and mobile ring 34 may additionally serve as protection from fraying and loose filaments 22 at the ends 26 and 28 of screen 20 and may comprise an assembly of concentric rings sandwiching ends 26 and 28 of screen 20.

Motion of mobile ring 34 may be limited by a stop 36 on one side of mobile ring 34 located toward balloon 12, and an extensible element 38 on the other side of mobile ring 34. Extensible element 38 may be fixed to catheter shaft 16 with the aid of a second fixed ring 32′ optionally configured like fixed ring 32.

Extensible element 38 optionally provides a refraction force to screen 20 upon the deflation of an inflated balloon and can be comprised of one or a combination of, but not limited to, an elastic tube, an extension of screen 20, a coil spring, or a number of extensible wires or rods.

Notably, either one of fixed ring 32 or mobile ring 34 (and any associated structure) may be oriented proximally or distally with respect to balloon 12 and/or the user. The same holds true with respect to the other embodiments herein.

FIGS. 7A and 7B depict an example embodiment of device 40, where similar action to device 30 is contemplated, but a different spring and mobile ring configuration is provided. As shown, a coil spring 48 either underlies or overlays screen 20 to accommodate the shortening of screen 20 upon the inflation of balloon 12. In addition, a mobile ring 44 is able to translate and allow spring compression until bottoming at an optional stop 46. In this embodiment, mobile ring 44 is set at the outer-most position relative to balloon 12. However, as above, a fixed ring 42 is provided at the side of balloon 12.

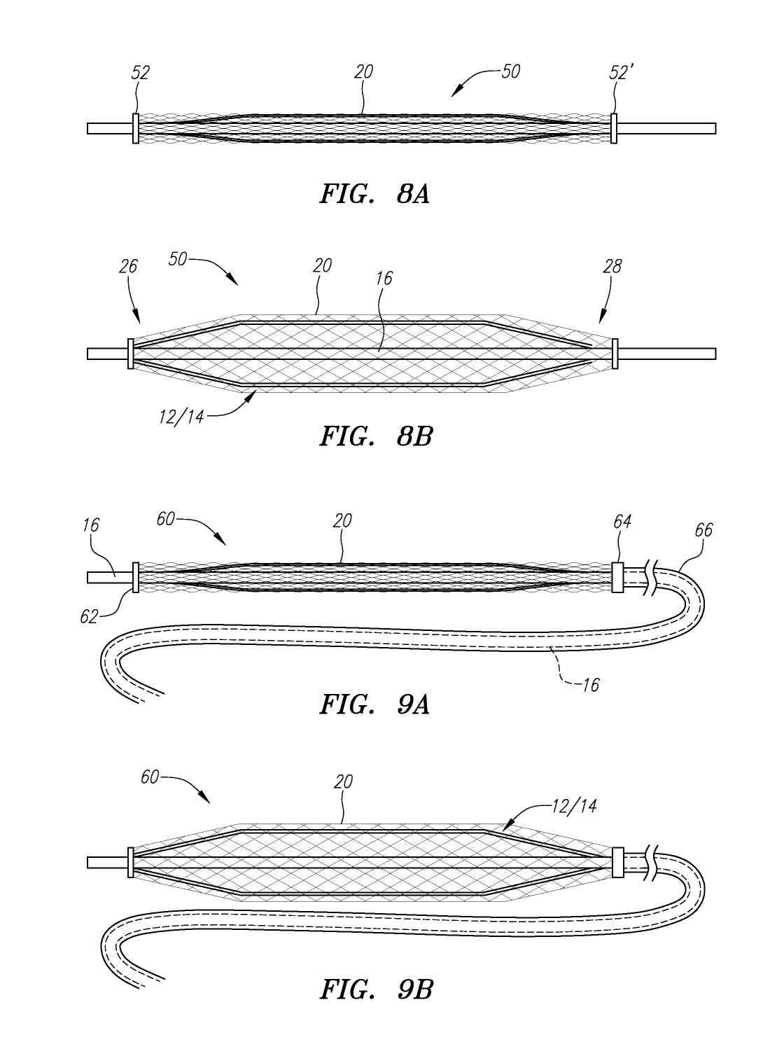

FIGS. 8A and 8B depict an example embodiment of device 50. Here, the mobile or translational elements are absent and both ends of screen 20 are fixed to catheter shaft 16 using fixed rings 52 and 52′. Again, screen 20 may be secured or coupled to the catheter by bonding (direct or indirect) by laser welding, contact welding, or any other technique known to those of ordinary skill in the art.

However secured, shortening of screen 20 (e.g., as resultant from changing braid angles) upon inflation of balloon 12 is optimally accommodated for by the bending or curving of the section of catheter shaft 16 between the two ends of screen 20. Such action may also provide restoring force to collapse screen 20 upon the deflation of the balloon 12.

Alternatively, if screen 20 is made up of suitably elastic material(s), these may stretch and accommodate the additional length required without causing significant curvature of the catheter tube. Such stretch can also provide restoring force to screen 20 upon balloon deflation.

In another example embodiment, restoring force is provided manually by the user. FIGS. 9A and 9B depict an example embodiment of device 60. Here, a fixed ring 62 attaches screen 20 at a distal end of catheter shaft 16 and a mobile ring 64 is attached to a proximal end of screen 20 together with an accessible extension 66 that runs to the proximal end of the device such that screen 20 can be restored manually to its closed pore state after deflation of balloon 12 by manual retraction of the extension outside the body of a patient.

The extension can take many forms, including but not limited, to a wire attached to mobile ring 64 and supported along the length of the catheter with aid of supporting rings or it can be in the form of a tube or sleeve that circumscribes the catheter tube and can move freely from it, as shown. Such a tube or sleeve may be an extension of (i.e., without an intermediate connection to) screen 20. The extension portion may be coated or overlaid with material, including a hydrophilic coating as typical in catheter construction.

Methods of use of the subject devices include not only their use in tracking to a target site while protecting a drug under the core-screen, options for inflation/deflation for drug delivery and withdrawal, but also manners of manipulating the core-screen as related to the above. The subject methods may also include the activity of drug transport and absorption. Indeed, any methodology implicit to operation of the devices discussed above forms inventive embodiments and may be explicitly claimed.

Further, it is to be appreciated that publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed. Also, all of the publications cited herein are incorporated by reference in their entireties.

It is also noted that all features, elements, components, functions, acts and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

In many instances entities are described herein as being coupled to other entities. It should be understood that the terms “coupled” and “connected” (or any of their forms) are used interchangeably herein and, in both cases, are generic to the direct coupling of two entities (without any non-negligible intervening entities) and the indirect coupling of two entities (with one or more non-negligible intervening entities). Where entities are shown as being directly coupled together, or described as coupled together without description of any intervening entity, it should be understood that those entities can be indirectly coupled together as well unless the context clearly dictates otherwise.

Reference to a singular item, includes the possibility that there is a plurality of the same items present. More specifically, as used herein and in the appended claims, the singular forms “a,” “an,” “said,” and “the” include plural referents unless specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element irrespective of whether a given number of elements are enumerated in the claim, or the addition of a feature could be regarded as transforming the nature of an element set forth in the claims. Except as specifically defined herein, all technical and scientific terms used herein are to be given as broad a commonly understood meaning as possible while maintaining claim validity.

Accordingly, while the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations (as referenced above, or otherwise) that define the inventive scope of the claims by features, functions, steps, or elements that are not within that scope.

What is claimed is:

1. A drug delivery device comprising:
a drug-coated balloon; and
a screen having proximal and distal ends,
wherein the screen ends, alone, are secured to the balloon, and
wherein the balloon is adapted to function clinically without support of the screen.

2. The device of claim 1, wherein the screen is in the form of a sleeve.

3. The device of claim 2, wherein the sleeve comprises a plurality of interlaced filaments able to move relative to one another.

4. The device of claim 3, wherein the sleeve comprises braided material.

5. The device of claim 1, wherein the screen is uncoated.

6. The device of claim 1, wherein at least one end of the screen is secured to the balloon by connection with a ring.

7. The device of claim 6, wherein both ends of the screen are secured to balloon.

8. The device of claim 6, wherein one end of screen is secured to the balloon by connection to a spring.

9. The device of claim 8, wherein the device is adapted so that the spring expands upon balloon inflation.

10. The device of claim 8, wherein the device is adapted so that the spring compresses upon balloon inflation.

11. The device of claim 6, further comprising an extension to a proximal end of the device attached to the proximal end of the screen.

12. A method of using a drug-eluting balloon in a lumen of a patient, comprising:
advancing a drug-coated balloon device through the lumen to a treatment site while the drug provided on a surface of the balloon is protected by a screen disposed over the drug and attached to the device;
inflating the balloon at the treatment site such that the screen expands and a plurality of spaces in the screen are opened; and
allowing the drug to transfer through the plurality of spaces to the target site.

13. The method of claim 12, wherein the balloon is inflated until the screen is in apposition with the target site.

14. The method of claim 12, wherein the screen is in apposition with plaque when the balloon is inflated.

15. The method of claim 12, further comprising partially deflating the balloon while remaining at the target site.

16. The method of claim 15, further comprising at least partially separating the balloon and screen.

17. The method of claim 16, wherein the screen is at least partially in apposition with the target site after the deflating.

18. The method of claim 17, further comprising continuing to allow the drug to transfer with the balloon partially deflated.

19. The method of claim 12, wherein the screen shortens relative to the balloon during balloon expansion.

20. The method of claim 12, further comprising fully deflating the balloon for withdrawal from the patient.

21. The method of claim 20, further comprising contracting the screen for withdrawal.

22. The method of claim 21, wherein the contracting is provided by elastic or spring action.

23. The method of claim 21, wherein the contracting is provided by retracting an extension to the device located outside of the patient.

24. A drug delivery device comprising:
a balloon carrying a drug, the balloon being transitionable between a contracted state and an at least partially expanded state; and
a screen adapted to reside over the balloon, the screen being transitionable between a contracted state and an at least partially expanded state, wherein the screen is adapted to protect the drug when the balloon and screen are in the contracted states, and wherein the screen is adapted to permit transfer of the drug to a target site through a plurality of spaces in the screen when the balloon and screen are in their at least partially expanded states.

25. The drug delivery device of claim 24, wherein the drug is coated on the outer surface of the balloon and wherein the drug breaks off of the balloon in the form of particulates.

26. The drug delivery device of claim 25, wherein the particulates are flakes.

27. The drug delivery device of claim 25, wherein the plurality of spaces in the screen are each larger than the particulates when the screen is in the at least partially expanded state for drug transfer.

28. The drug delivery device of claim 24, wherein the screen is adapted to substantially prevent transfer of the drug therethrough when the screen is in the contracted state.
